(19) United States
(12) Patent Application Publication
(10) Pub. No.: US 2026/0146048 A1
(43) Pub. Date: May 28, 2026

(54) NOVEL BISINDOLYLMALEIMIDE KINASE INHIBITORS

(71) Applicant: 4M Therapeutics Inc., Skillman, NJ (US)

(72) Inventors: Pablo Lapuerta, Skillman, NJ (US); Daniel E. Levy, San Mateo, CA (US)

(73) Assignee: 4M Therapeutics Inc., Skillman, NJ (US)

(21) Appl. No.: 19/395,194

(22) Filed: Nov. 20, 2025

Related U.S. Application Data
(60) Provisional application No. 63/725,508, filed on Nov. 26, 2024.

(51) Int. Cl. C07D 498/22; A61K 31/19; A61K 31/407; A61K 31/4439; A61K 31/46; A61K 31/496; A61K 31/517; A61K 31/53; A61K 31/5513; A61K 31/554; A61K 33/00

(52) U.S. Cl. CPC C07D 498/22; A61K 31/19; A61K 31/407; A61K 31/4439; A61K 31/46; A61K 31/496; A61K 31/517; A61K 31/53; A61K 31/5513; A61K 31/554; A61K 33/00

(57) ABSTRACT

Aspects of this invention are related to novel compounds of Formula I and compositions thereof that are bisindolylmaleimide kinase inhibitors with activity against GSK-3, PKC, RSK1, and CDK4/6. Some aspects of the invention relate to novel compositions having a better therapeutic window than previous bisindolylmaleimide kinase inhibitors, a profile more suitable to therapeutic use.

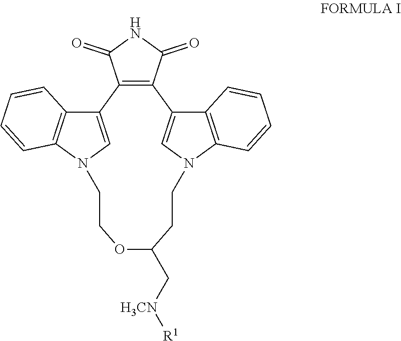

### FIELD OF THE INVENTION

Aspects of this invention are related to novel compositions for treating neurological disease or psychiatric disorders, including Alzheimer's disease, bipolar disorder, depression, neuroinflammation, autism spectrum disorders, or other conditions, including metabolic disorders, cardiovascular disorders, eye diseases and cancers, where modulation of GSK-3 signaling, PKC signaling, RSK1 signaling, or CDK4/6 signaling is clinically useful.

### BACKGROUND OF THE INVENTION

Ruboxistaurin is a bisindolylmaleimide that has been shown to modulate signaling of glycogen synthase kinase 3 (GSK-3) and to inhibit protein kinase C.

GSK-3 inhibitors have been proposed as a treatment for subjects having a neurological disease and/or psychiatric disorder, including Alzheimer's disease, frontotemporal dementia, behavioral complications of dementia, bipolar disorder, depression, post-traumatic stress disorder, schizophrenia, Parkinson's disease, neuroinflammation, autism spectrum disorder SYNGAP-1 related disorders, Fragile X syndrome, Pitt Hopkins syndrome, Rett syndrome, traumatic brain injury, stroke, acute spinal cord injury, amyotrophic lateral sclerosis (ALS), multiple sclerosis (MS), neurofibromatosis type 1, neuronal ceroid lipofuscinosis, chronic pain, neuropathic pain, chemotherapy-induced neuropathy, and/or chemotherapy-induced cognitive impairment.

Inhibitors of GSK-3 are known to increase the expression of WNT proteins, thereby enhancing a pathway in regenerative medicine that has been broadly proposed to treat neurological and psychiatric disorders and reduce neuroinflammation. GSK-3 inhibition or enhancement of WNT signaling has been linked to the potential treatment of type 2 diabetes, diabetic nephropathy, chronic kidney disease, atherosclerosis, alopecia, osteoarthritis, osteoporosis, alcoholic hepatitis, inflammatory bowel disease, wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, Sjogren's syndrome, sensorineural hearing loss, conductive hearing loss, polycystic kidney disease, focal segmental glomerulosclerosis, chronic obstructive pulmonary disease (COPD), idiopathic pulmonary fibrosis, short bowel syndrome, melanoma, pancreatic cancer, prostate cancer, colon cancer, leukemia, and septic shock.

As a GSK-3 inhibitor, the use of ruboxistaurin has been proposed as a monotherapy for treating bipolar disorder, or in combination with lithium, or in combination with other bipolar disorder treatments.

As a protein kinase C inhibitor, ruboxistaurin has been proposed for treating conditions associated with diabetes mellitus, diabetic nephropathy, diabetic neuropathy, diabetic retinopathy, ischemia, inflammation, pulmonary hypertension, congestive heart failure, cardiovascular disease, dermatological disease, cancer and GM2 gangliosidosis.

Ruboxistaurin is metabolized into N-desmethyl ruboxistaurin, which is a more potent and selective inhibitor of GSK-3p and retains activity against PKC. N-desmethyl ruboxistaurin has also been proposed for treating conditions including cancers where inhibition of RSK1 or CDK4/6 is clinically useful. RSK1 inhibition has been proposed as a treatment for breast cancer, ovarian cancer, prostate cancer, lung cancer, hepatocellular carcinoma, colorectal cancer, melanoma, osteosarcoma, leukemia, or bladder cancer. CDK4/6 inhibition has been proposed as a treatment for breast cancer, liposarcoma, lung cancer, glioblastoma, melanoma, pancreatic cancer, prostate cancer, colon cancer, ovarian cancer, colorectal cancer, bladder cancer, hepatocellular carcinoma, osteosarcoma, germ cell tumors, and advanced solid tumors, lymphoma, leukemia and other hematologic malignancies

Ruboxistaurin pharmacokinetics include a high peak to trough ratio for inhibition of GSK-3 and PKC, raising the potential for toxicity. And ruboxistaurin levels can be increased by drugs that inhibit CYP3A4 metabolism.

### BRIEF SUMMARY OF THE INVENTION

Compounds have now been developed that overcome these shortcomings of ruboxistaurin. Compounds and compositions of the present invention are provided that surprisingly and unexpectedly have been found to have a better therapeutic window than ruboxistaurin. One aspect of the invention is directed to a bisindolylmaleimide having the structure of Formula I

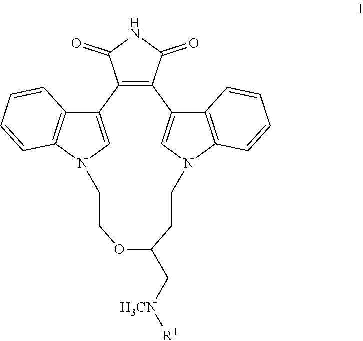

or a pharmaceutically acceptable salt thereof; wherein,
R¹ is —(C═O)—X—(CH₂)ₙ—R⁷ or —(C═O)—R³;
R² is H, —O—(C═O)ₒ—R⁴, —N(—(C═O)ₙ—R⁴))₂, S—(C═O)—R⁴, —(C═O)—OR⁴;
R³ is —Y—R¹, wherein R¹ is attached to Y at a position adjacent to the position where Y is attached to R¹;
each R⁴ is independently H or C₁-C₆ alkyl;
R⁵ is —(CH₂)ₚ—R⁶;
R⁶ is —C—(C═O)_{q}—R⁷, N((C═O)_{q}—R⁷))₂, —S—(C═O), R⁷, —(C═O)—OR⁷;
each R⁷ is independently H or C₁-C₆ alkyl;
R⁸ is H or the residue of a naturally occurring amino acid;
each R⁹ is independently H or C₁-C₆ alkyl;
X is —O— or —CHR⁸;
Y is a 5-10 membered aryl or bicycloaryl group comprising 0-3 heteroatoms selected from N, O and S and optionally substituted with 1-3 groups selected from C₁-C₆ alkyl, F, Cl, Br, I, —OR⁹, —N(R⁹)₂, —SR⁹, —(C═O)OR⁹, —(C═O)N(R)₂, ON, NO₂ and —(C═O)—R⁹;
n is 0 or an integer from 1-6;
each o is independently 0 or 1;
p is 0 or an integer from 1-3;
each q is independently 0 or 1; and,
each stereogenic center is independently R or S.

A kinase inhibitor according to the present invention can also have any one of the structures 3, 5, 6, 7, 8, 10, or 11.

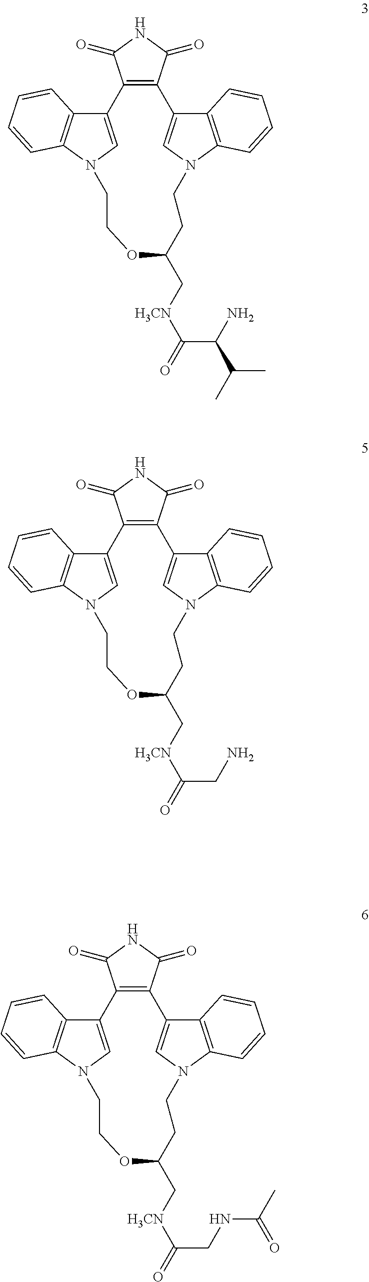

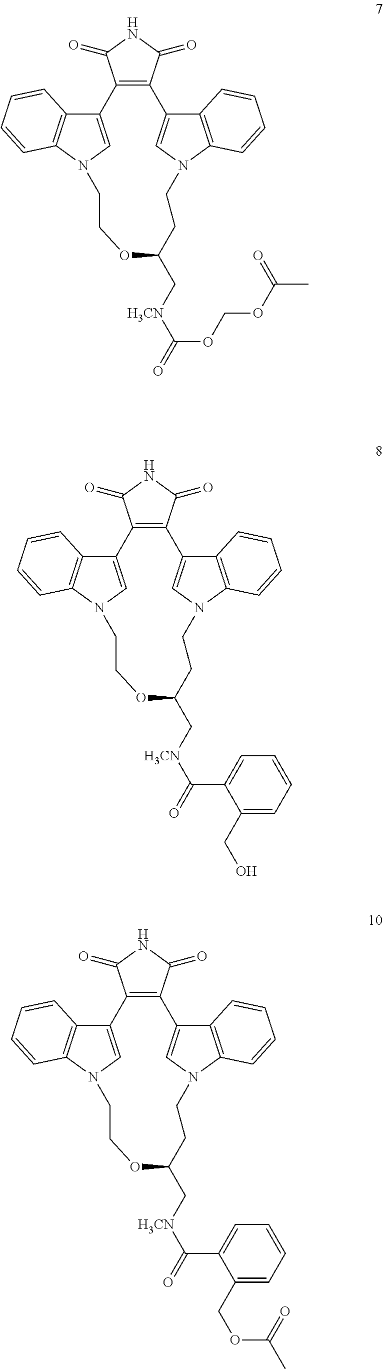

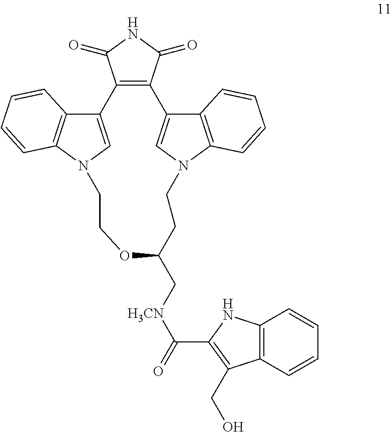

Other aspects of the invention include the pharmaceutical compositions and therapeutic methods disclosed herein using the compounds of the invention.

### BRIEF DESCRIPTION OF THE DRAWINGS

FIG. 1 shows the QT interval on an electrocardiogram (ECG) tracing, which describes that portion of the cardiac contraction cycle that starts with contraction of the left ventricle and ends with the relaxation of the left ventricle.

FIG. 2 shows a diagram of the therapeutic window for a drug on a graph of % maximum effect versus drug concentration.

FIG. 3 displays the chemical structure of ruboxistaurin.

FIG. 4 displays the chemical structure of N-desmethyl ruboxistaurin.

FIG. 5 shows a graph of the mean plasma concentration versus time profile following single-dose administration of 32 mg ruboxistaurin in healthy subjects, illustrating a high peak to trough ratio. Results in the insert are in the fed state.

### DETAILED DESCRIPTION

Unless otherwise defined, all terms used in disclosing the invention, including technical and scientific terms, have the meaning as commonly understood by one of ordinary skill in the art to which this invention belongs.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” can indicate a range of 9% to 11%, and “about 20” can mean from 18 to 22. Preferably “about” includes up to plus or minus 6% of the indicated value. Alternatively, “about” includes up to plus or minus 5% of the indicated value, Other meanings of “about” may be apparent from the context, such as rounding off, so, for example “about 1” can also mean from 0.5 to 1.4.

The term “pharmaceutically acceptable salt” of a compound means a salt that is pharmaceutically acceptable and that possesses the desired pharmacological activity of the parent compound. It is understood that the pharmaceutically acceptable salts are non-toxic. Such salts include acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Additional information on suitable pharmaceutically acceptable salts can be found in Remington's Pharmaceutical Sciences, 17th ed., Mack Publishing Company, Easton, PA, 1985, which is incorporated herein by reference.

As used herein, the term “therapeutically effective amount” means an amount of compound of the present invention which is capable of alleviating the symptoms of the various pathological conditions herein described. The specific dose of a compound administered according to this invention will, of course, be determined by the particular circumstances surrounding the case including, for example, the compound(s) administered, the route of administration, the state of being of the patient, and the pathological condition being treated. Dosing can be once per day, or administered in multiple sub-doses per day, e.g., two, three, or more doses per day,

The effective dose of the novel bisindolylmaleimide, or pharmaceutically acceptable salt, solvate or polymorph thereof, is about 20 to about 200 mg once daily, or about 10 to about 100 mg twice daily for monotherapy. A pharmaceutical composition of the bisindolylmaleimide, or pharmaceutically acceptable salt, solvate or polymorph thereof, further comprises at least one pharmaceutically acceptable adjuvant or excipient. For combination therapy, a sub-effective dose of the bisindolylmaleimide, or pharmaceutically acceptable salt, solvate or polymorph thereof, is about 8 to about 40 mg once daily, or about 4 to about 20 mg twice daily. When the bisindolylmaleimide is combined with lithium, a sub-effective dose of lithium can be about 60 mg to about 600 mg once daily, or about 30 mg to about 300 mg twice daily. This sub-effective dose of lithium can spare the kidney damage typically caused by lithium treatment. The once daily effective dose of the bisindolylmaleimide, or pharmaceutically acceptable salt, solvate, or polymorph thereof, can be about 20 or about 40 or about 60 or about 80 or about 100 or about 120 or about 140 or about 160 or about 200 mg. The twice daily effective dose of the bisindolylmaleimide, or pharmaceutically acceptable salt, solvate or polymorph thereof, can be about 10 or about 20 or about 30 or about 40 or about 50 or about 60 or about 70 or about 80 or about 100 mg. The once daily sub-effective dose of the bisindolylmaleimide, or pharmaceutically acceptable salt, solvate, or polymorph thereof, can be about 8 or about 12 or about 16 or about 20 or about 24 or about 28 or about 32 or about 36 or about 40 mg. The twice daily sub effective dose of Bisindolylmaleimide, or pharmaceutically acceptable salt, solvate or polymorph thereof, can be about 4 or about 8 or about 6 or about 8 or about 10 or about 12 or about 14 or about 16 or about 18 or about 20 mg.

Ruboxistaurin has been investigated in several clinical trials for the treatment of diabetes mellitus and its complications, including diabetic retinopathy, diabetic neuropathy, and diabetic nephropathy. See A. Girach, US Patent Publication No. 2008/0096923, incorporated herein by reference in its entirety. Its safety has been described as supporting its long-term use, with a lower incidence of serious adverse events than placebo. Further development of ruboxistaurin has been encouraged because of its clinical safety profile, and because of growing interest in the use of treatments that can inhibit GSK-3.

However, careful review of ruboxistaurin clinical and preclinical data has identified its potential to prolong the QT interval in human subjects, and this can increase the risk of dangerous cardiac arrhythmias, particularly in the case of an accidental or intentional overdose of ruboxistaurin, or when administered with other drugs that prolong the QT interval, or when very high ruboxistaurin levels are induced by co-administration of a drug inhibiting CYP3A4.

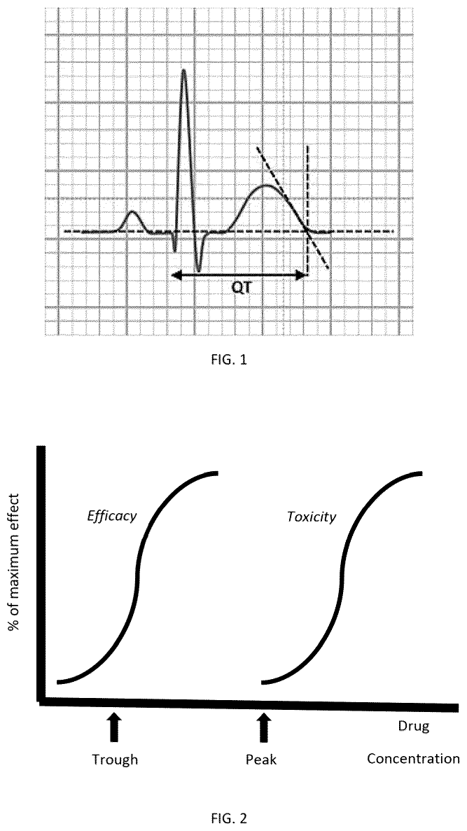

Evidence for these potential risks lie in the Withdrawal Assessment Report prepared by the European Medicines Agency (EMA). A Marketing Authorization Application for ruboxistaurin had been submitted to the European Medicines Agency, and the application was withdrawn, prompting the report. The Withdrawal Assessment Report showed that ruboxistaurin inhibits hERG (a potassium ion channel known for its contribution to electrical activity of the heart), This preclinical assessment is commonly used to identify compounds with a potential risk for QT prolongation. See FIG. 1

The pharmacokinetics of a compound are also important in considering safety. The potential for arrhythmias relates to the maximum plasma concentration of the compound. This has been observed in ECG studies where the maximum prolongation of the QT interval is shown near the time of maximum concentration of study drug.

A lower peak concentration (in relation to the trough concentration) is generally desirable as a principle of drug development because it keeps drug levels within the therapeutic window. There is a dose response for efficacy and a dose response for toxicity. A lower peak/trough ratio can help keep a drug concentration at levels that provide efficacy without being high enough to cause toxicity. See FIG. 2.

Ruboxistaurin 32 mg has a high peak concentration of about 90 nmol/L compared to its trough concentration of about 5 nmol/L (a peak trough ratio of about 13, consistent with a half-life of less than 6 hours). This raises the risk for several toxicities, of which worsening glucose control and elevations in creatine kinase were of concern for the European Medicines Agency.

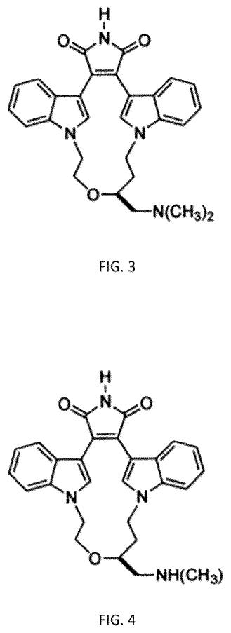

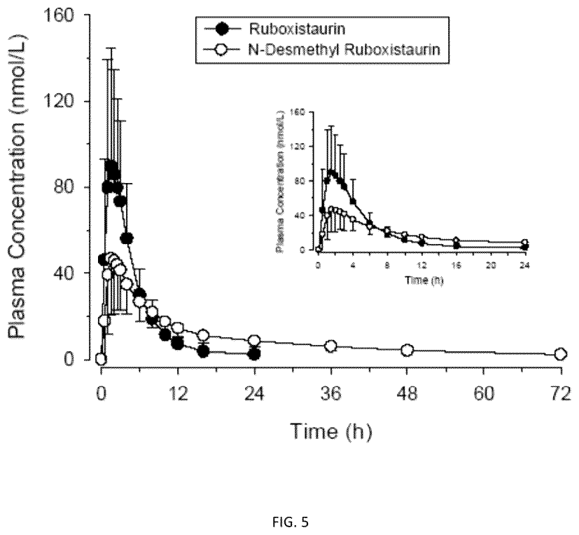

A third consideration affecting the peak concentration of a drug is potential interactions with other drugs. Ruboxistaurin is metabolized by CYP3A4, which converts ruboxistaurin to N-desmethyl ruboxistaurin. See FIGS. 3 and 4. N-desmethyl ruboxistaurin is an attractive metabolite because it is more potent, selective, stable, with a lower potential for CYP3A4 interactions than ruboxistaurin.

A new drug can be an important alternative to ruboxistaurin if it is metabolized into N-desmethyl ruboxistaurin, and if prior to its metabolism it has at least one or more of the following features: a similar or lower inhibition of hERG, a lower peak concentration during the dosing interval (better pharmacokinetics), a lower potential for drug-drug interactions, or a lower potential for toxicity related to kinase inhibition. An aspect of this invention is directed to using a novel bisindolylmaleimide, which has these features, in situations where inhibition of GSK-3, PKC, RSK1, or CDK4/6 is clinically useful.

The risk of potential toxicity can be further reduced by administering the novel bisindolylmaleimide together with lithium. It has been shown that lithium with another GSK-3 inhibitor has synergy in treating bipolar disorder in an animal model. A desired amount of GSK-3 inhibition can be achieved with a lower concentration of the novel bisindolylmaleimide if it is administered in conjunction with lithium. Thus, the bisindolylmaleimide, or its pharmaceutically acceptable salt, solvate or polymorph, can be administered in combination with lithium for bipolar disorder, or other conditions where inhibition of GSK-3, protein kinase C, or both, is useful.

While this combination can lower the dose of the novel bisindolylmaleimide, it can also lower the dose of lithium required for the treatment of bipolar disorder, Alzheimer's disease, and other conditions where GSK-3 inhibition by lithium is clinically desirable, and thereby improve safety. The dose of bisindolylmaleimide in the combination can be a dose lower than what would be needed as monotherapy (a sub-effective dose), and the dose of lithium in the combination can be a dose lower than what would be needed as monotherapy (a sub-effective dose). The combination can be used to provide efficacy in subjects non-responsive to lithium or intolerant of lithium at standard doses. The bisindolyimaleimide can be used to provide additional efficacy in subjects who have only a partial response to lithium, as an alternative to higher lithium doses.

Furthermore, ruboxistaurin has been proposed in combination with valproic acid, lamotrigine, carbamazepine, gabapentin, and topiramate for the treatment of a neurological disease and/or a psychiatric disorder. With superior pharmacokinetics and a lower potential for toxicity than ruboxistaurin, the novel bisindolylmaleimide can be used in combination with valproic acid, lamotrigine, quetiapine, olanzapine, risperidone, aripiprazole, lurasidone, cariprazine, asenapine, carbamazepine, and xanomeline, or with the combination of xanomeline with trospium.

Atypical antipsychotics and muscarinic agonists not known to prolong the QT interval, which include olanzapine, risperidone, aripiprazole, iloperidone lurasidone and xanomeline, have also been used for the treatment of neurological disease and/or psychiatric disorders including bipolar disorder, depression, Parkinson's disease and schizophrenia. The novel bisindolylmaleimide can be combined with these atypical antipsychotics and muscarinic agonists to treat these conditions.

Furthermore, a response to either the bisindolylmaleimide or the combination of the bisindolylmaleimide with lithium can serve to establish a diagnosis of bipolar disorder and other conditions where GSK-3 inhibition is clinically useful. In central nervous system disorders, positron emission tomography (PET) of GSK-3 beta activity and blood-based biomarkers are being developed as diagnostics. The novel bisindolylmaleimide, alone or in combination with lithium, may be administered to subjects with excess GSK-3 beta activity on PET or a blood-based biomarker in order to treat central nervous system disorders, and a reduction in GSK-3 beta activity on PET or a blood-based biomarker after administration of the bisindolylmaleimide can support its use (alone or in combination with lithium or other agents) as an appropriate therapy administered at a suitable dose.

The presently disclosed compositions and treatment methods are relevant wherever GSK-3 inhibition is clinically useful, including psychiatric and neurological disorders, such as bipolar disorder, depression, Alzheimer's disease, autism spectrum disorder, Fragile X syndrome, SYNGAP-1 related disorders, Rett syndrome, Pitt Hopkins syndrome, traumatic brain injury, stroke, acute spinal cord injury, schizophrenia, Parkinson's disease, amyotrophic lateral sclerosis (ALS), and multiple sclerosis (MS). These compositions and treatment methods are also relevant to indications where ruboxistaurin has been applied as a protein kinase C inhibitor, including diabetes mellitus, diabetic nephropathy, diabetic neuropathy, diabetic retinopathy, ischemia, inflammation, cardiovascular disease, pulmonary hypertension, congestive heart failure, dermatological disease, cancer and GM2 gangliosidosis. They are relevant to conditions where GSK-3 inhibition and or enhancement of WNT signaling have been proposed, including alopecia, osteoarthritis, osteoporosis, alcoholic hepatitis, inflammatory bowel disease, wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, Sjdgren's syndrome, sensorineural hearing loss, conductive hearing loss, schizophrenia, Parkinson's disease, polycystic kidney disease, focal segmental glomerulosclerosis, chronic obstructive pulmonary disease, idiopathic pulmonary fibrosis, short bowel syndrome, melanoma, pancreatic cancer, prostate cancer, colon cancer, leukemia, septic shock, and ischemia/reperfusion injury,

The presently disclosed compositions and treatment methods also have use in veterinary applications for improving the health and well-being of livestock and companion animals by treating any of the foregoing indications that occur in animals.

It will be understood by those of ordinary skill in the art that numerous and various modifications can be made without departing from the spirit of the present invention. Therefore, it should be clearly understood that the various embodiments of the present invention described herein are illustrative only, and they are not intended to limit the scope of the present invention.

Therefore. one aspect of the invention is directed to bisindolylmaleimides having the structure of Formula I

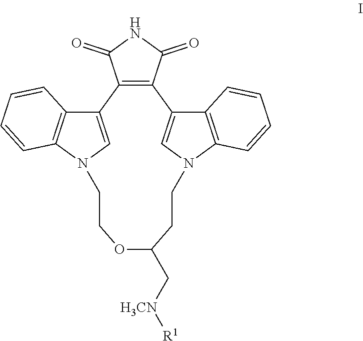

or a pharmaceutically acceptable salt thereof; wherein,
R¹ is —(C═O)—X—(CH₂)ₙ—R² or —(C═O)—R³;
R² is H, —O—(C═O)ₒ—R⁴, —N(—(C═O)ₒ—R⁴))₂, —S—(C═O)—R⁴, —(C═O)—OR⁴;
R³ is —Y—R⁵, wherein R⁵ is attached to Y at a position adjacent to the position where Y is attached to R¹;
each R⁴ is independently H or C₁-C₆ alkyl;
R⁵ is —(CH₂)ₚ—R⁶;
R⁶ is —O—(C═O)_{q}R⁷, —N(—(C═O)_{q}—R⁷))₂, —S—(C═O)ₙ—R⁷, —(C═O)—OR⁷;
each R⁷ is independently H or C₁-C₆ alkyl;
R⁸ is H or the residue of a naturally occurring amino acid;
each R⁸ is independently H or C₁-C₆ alkyl;
X is —O— or —CHR⁸—;
Y is a 5-10 membered aryl or bicycloaryl group comprising 0-3 heteroatoms selected from N, O and S and optionally substituted with 1-3 groups selected from C₁-C₆ alkyl, F, Cl, Br, I, —OR⁹, —N(R⁹)₂, —SR⁹, —(C═O)OR⁹, —(C═O)N(R⁹)₂, CN, NO₂ and —(C═O)—R⁹;
n is 0 or an integer from 1-6;
each o is independently 0 or 1;
p is 0 or an integer from 1-3;
each q is independently 0 or 1; and,
each stereogenic center is independently R or S.

Kinase inhibitors according to the present invention can also have any one of the structures:

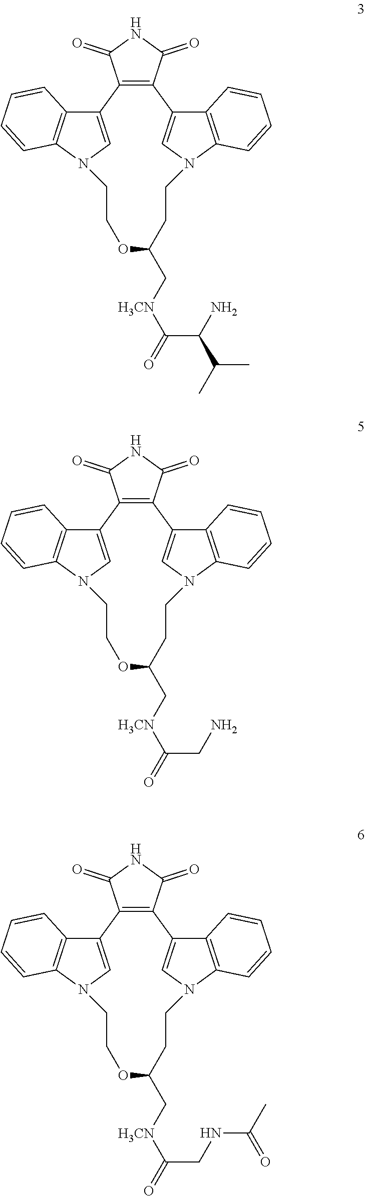

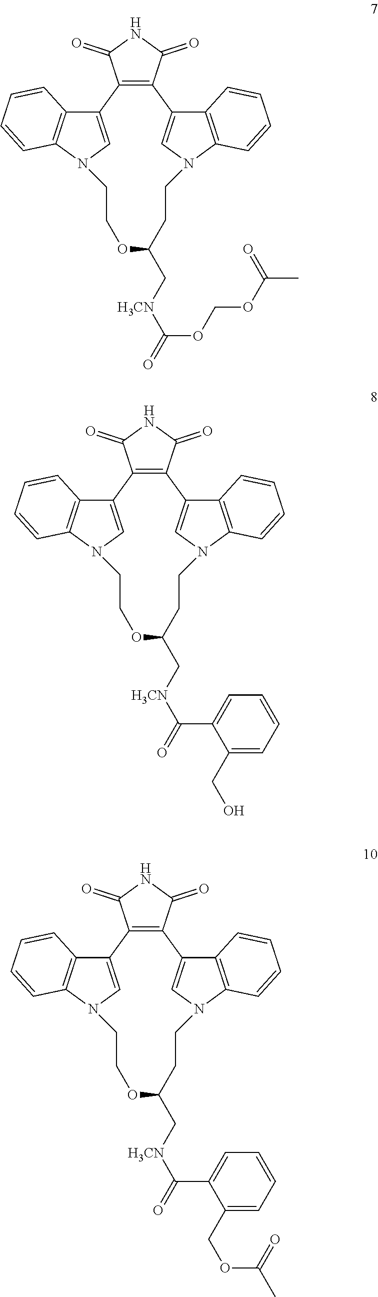

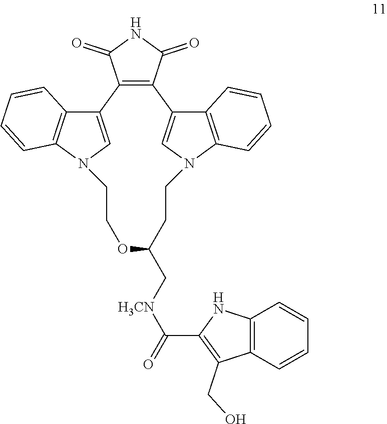

### Examples

The above structures are available according to the following Schemes. As illustrated in Scheme 1, N-desmethyl ruboxistaurin, 1, is coupled with N-Boc-valine utilizing HBTU as a coupling reagent to give compound 2. The Boc group is subsequently cleaved on reaction with hydrochloric acid giving compound 3. One of ordinary skill in the art will recognize that the Boc protecting group can be replaced with alternate protecting groups. Additionally, one of ordinary skill in the art will recognize that alternates to HBTU are available for generation of the amide bond, Finally, one of ordinary skill in the art will recognize alternate conditions equally useful for cleavage of a Boc protecting group.

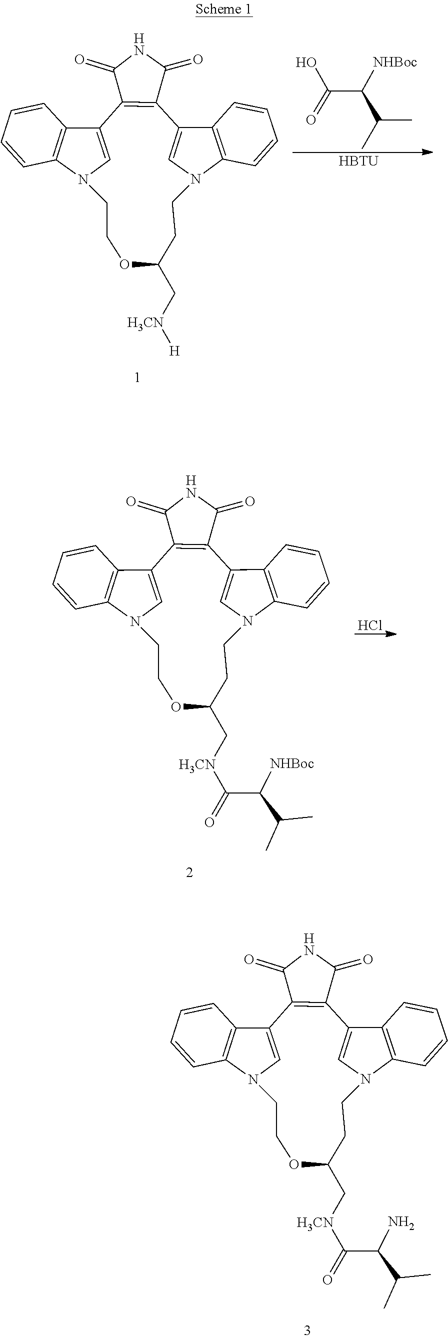

Mirroring the chemistry illustrated in Scheme 1, compound 5 is available as shown in Scheme 2 using similar chemistry to couple N-Boc glycine with N-desmethyl ruboxistaurin, 1.

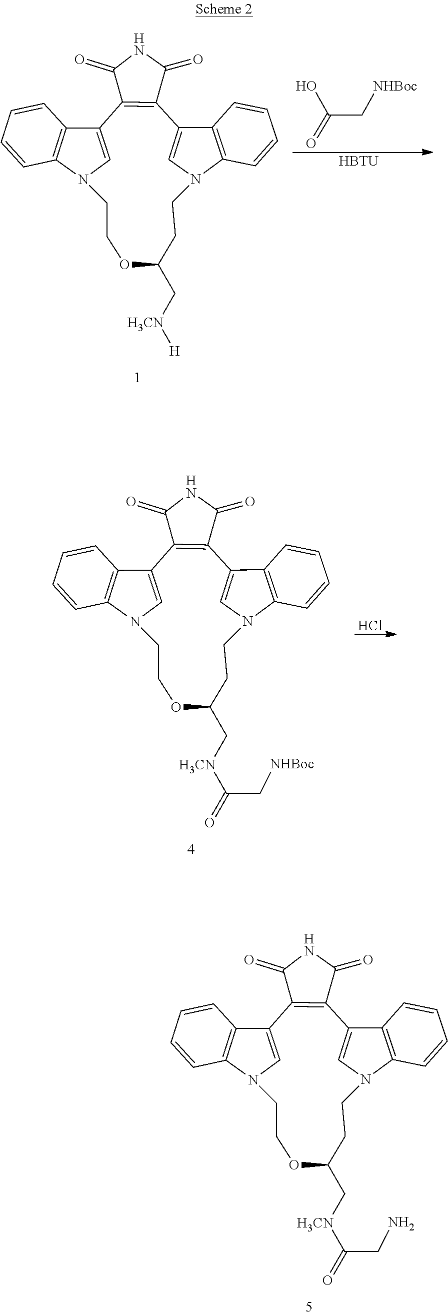

Scheme 3 further illustrates the generality of the chemistry used to prepare compounds 3 and 5 by utilizing the same coupling method to couple N-desmethyl ruboxistaurin, 1, with N-acetylglycine giving compound 6.

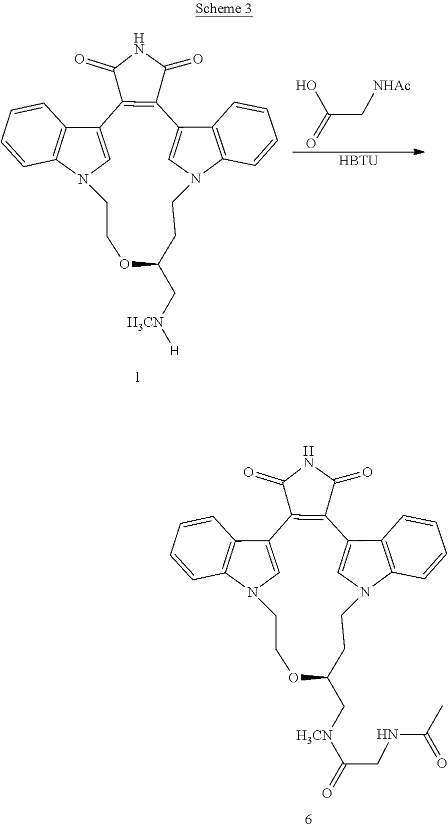

Departing from standard amide forming reagents, activated carbonyl species such as chloroformates and acid chlorides are also useful in the preparation of amide and carbamate functional groups. One of ordinary skill in the art will recognize that additional activated carbonyl species including, but not limited to, N-acylimidazoles, N-hydroxysuccinimidyl esters and pentafluorophenyl esters are also useful.

Scheme 4 illustrates use of chloroformates in the preparation of compound 7. As illustrated, N-desmethyl ruboxistaurin, 1, is shown coupling with the a chloroformate with diisopropyl ethylarnine giving the target compound. One of ordinary skill in the art will recognize that amine bases in general are useful for this type of coupling reaction. In addition, one of ordinary skill in the art will recognize the utility of inorganic bases including, but not limited to, sodium hydride, sodium hydroxide, sodium bicarbonate, potassium carbonate, cesium carbonate and the like as alternatives to amine bases.

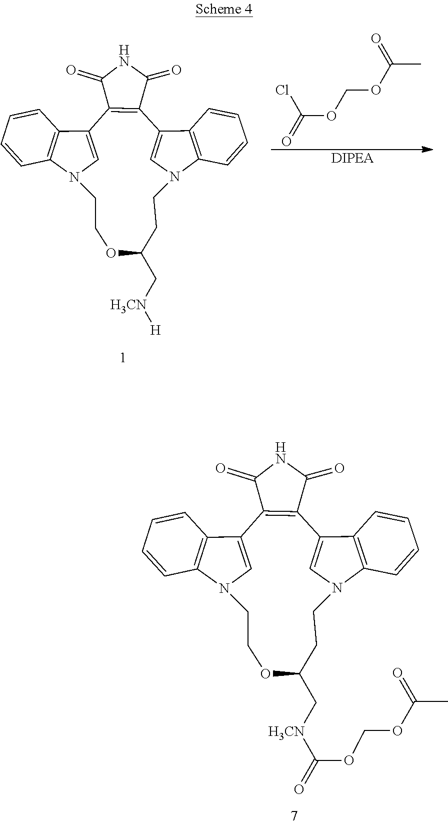

Amides are also known to be available on direct reaction of amines with esters or lactones. As illustrated in Scheme 5, N-desmethyl ruboxistaurin, 1, is shown reacting with a lactone and a Lewis acid catalyst giving target compound 8. One of ordinary skill in the art will recognize that alternate Lewis acids are available for facilitating this type of amide formation.

In addition, Scheme 5 illustrates an alternate route to target compound 8 utilizing an acid chloride approach. In this case, compound 9 is formed as an intermediate and a reduction reaction is required following the initial amide formation to reach target compound 8. One of ordinary skill in the art will recognize that sodium borohydride is not the only useful reducing agent for this reaction and that many alternatives including, but not limited to, lithium borohydride, sodium cyanoborohydride and sodium triacetoxyborohydride are available.

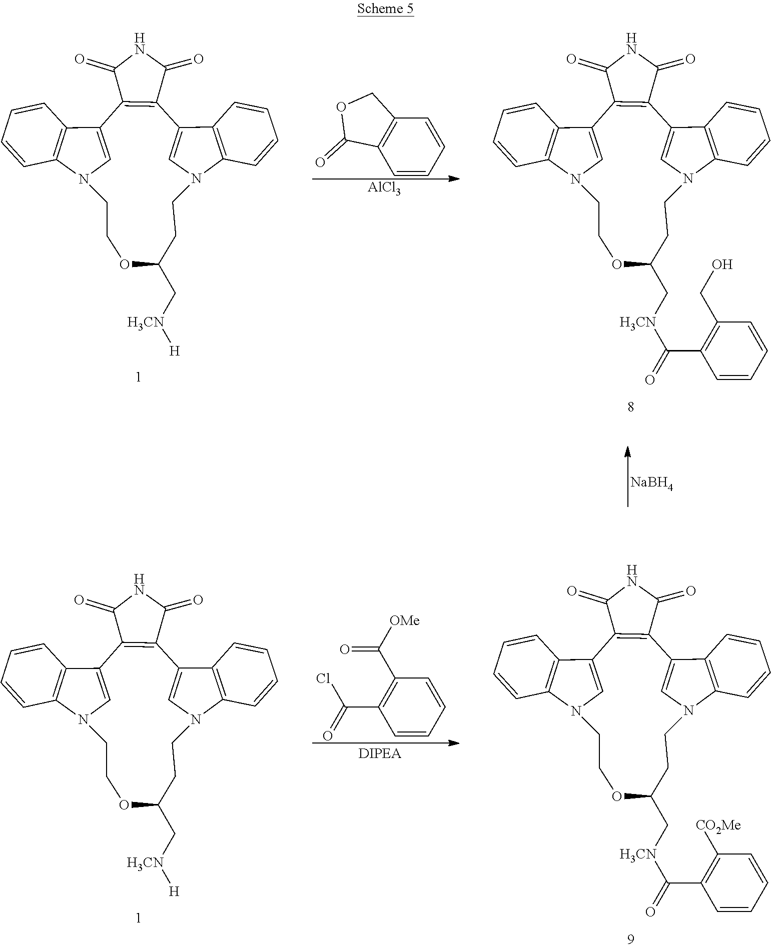

Scheme 6 illustrates preparation of compound 10 utilizing HATU as the amide-forming reagent. Similarly, Scheme 7 illustrates preparation of compound 11 utilizing BOP and diisopropyl ethylamine for the reaction conditions. Again, these are direct modifications of N-desmethyl ruboxistaurin, 1. One of ordinary skill in the art will recognize that all options for formation of amides discussed herein apply equally to these examples.

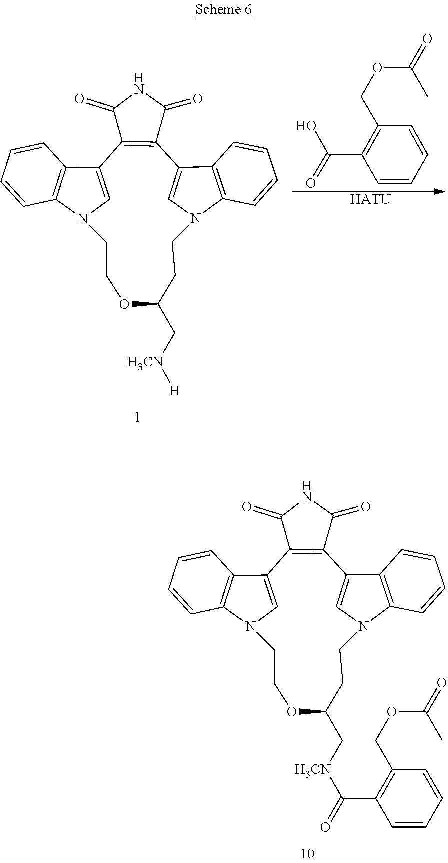

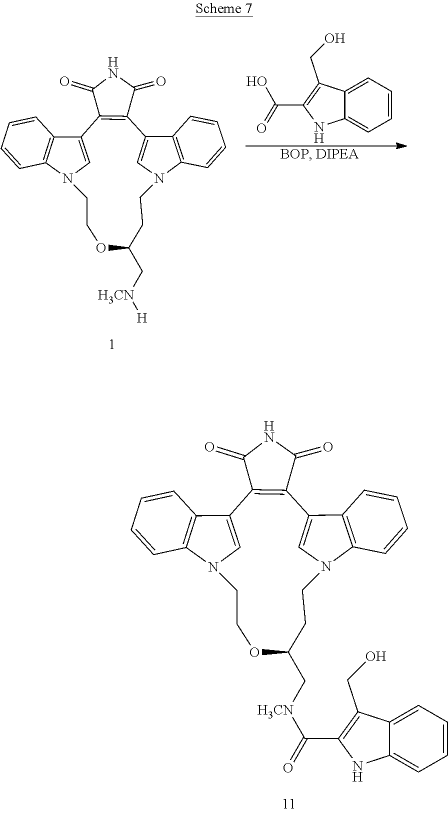

We claim:

1. A bisindolylmaleimide having the structure of Formula I 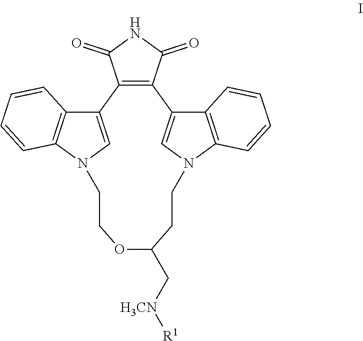
or a pharmaceutically acceptable salt thereof, wherein,
R¹ is —(C═O)—X—(CH₂)ₙ—R² or —(C═O)—R³;
R² is H, —O—(C═O)ₒ—R⁴, —N(—(C═O)ₒ—R⁴))₂, —S—(C═O)ₒ—R⁴, —(C═O)—OR⁴;
R³ is —Y—R⁵, wherein R⁵ is attached to Y at a position adjacent to the position where Y is attached to R¹;
each R⁴ is independently H or C₁-C₆ alkyl;
R⁵ is —(CH₂)ₚ—R⁶;
R⁶ is —O—(C═O)_{q}—R⁷, —N(—(C═O)_{q}—R⁷))₂, —S—(C═O)_{q}—R⁷, —(C═O)—OR⁷;
each R⁷ is independently H or C₁-C₆ alkyl;
R⁸ is H or the residue of a naturally occurring amino acid;
each R⁹ is independently H or C₁-C₆ alkyl;
X is —O— or —CHR⁸—;
Y is a 5-10 membered aryl or bicycloaryl group comprising 0-3 heteroatoms selected from N, O and S and optionally substituted with 1-3 groups selected from C₁-C₆ alkyl, F, Cl, Br, I, —OR⁹, —N(R⁹)₂, —SR⁹, —(C═O)OR⁹, —(C═O)N(R⁹)₂, CN, NO₂ and —(C═O)—R⁹;
n is 0 or an integer from 1-6;
each o is independently 0 or 1;
p is 0 or an integer from 1-3;
each q is independently 0 or 1; and,
each stereogenic center is independently R or S.

2. The bisindolylmaleimide of claim 1, having any one of the structures 3, 5, 6, 7, 8, 10, or 11.

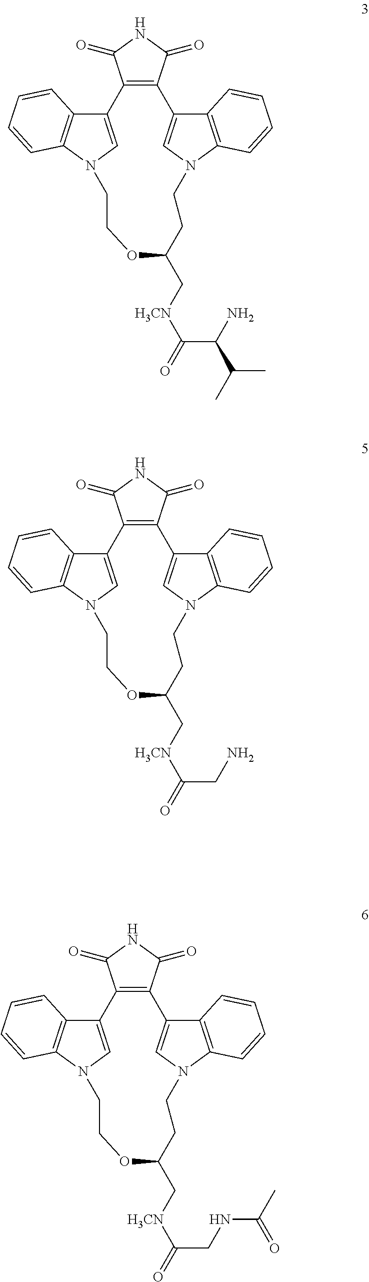

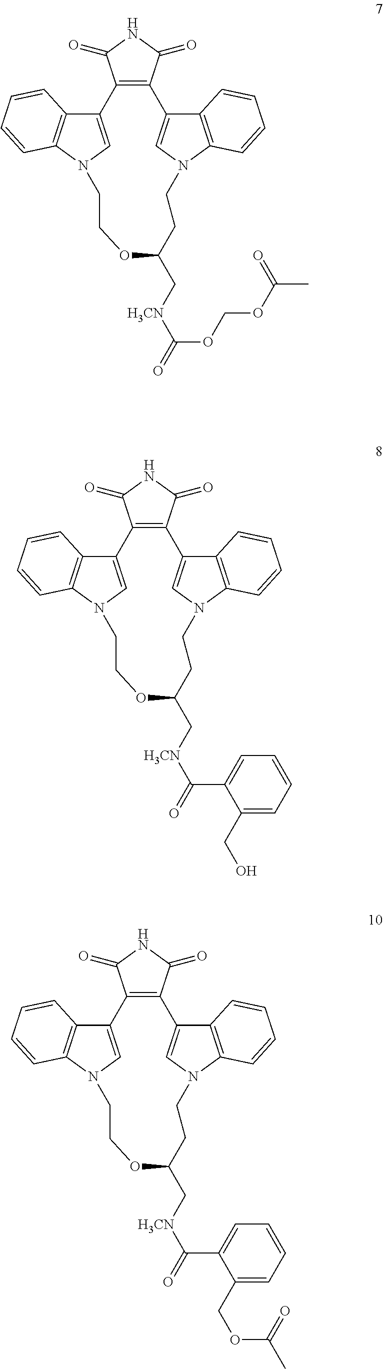

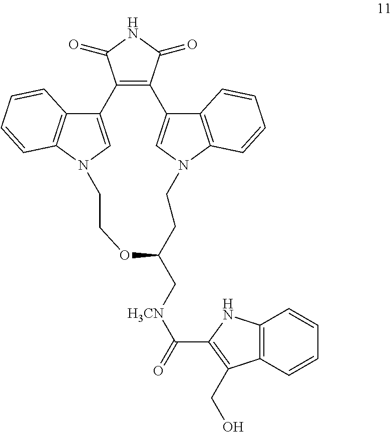

3. A method of treating a disorder comprising aberrant signaling of GSK-3, the method comprising administering to a subject in need thereof a therapeutically effective dose of a compound of claim 1, wherein the compound is administered in an amount of about 20 to about 200 mg once daily of the compound, or about 10 to about 100 mg twice daily.

4. The method of claim 3, wherein the subject has a neurological disease and/or psychiatric disorder selected from the group consisting of Alzheimer's disease, frontotemporal dementia, behavioral complications of dementia, bipolar disorder, depression, post-traumatic stress disorder, schizophrenia, Parkinson's disease, neuroinflammation, autism spectrum disorder, synGAP1 disorder, Fragile X syndrome, Pitt Hopkins syndrome, Rett syndrome, traumatic brain injury, stroke, acute spinal cord injury, amyotrophic lateral sclerosis (ALS), multiple sclerosis (MS), neurofibromatosis type 1, neuronal ceroid lipofuscinosis, chronic pain, neuropathic pain, chemotherapy-induced neuropathy, and/or chemotherapy-induced cognitive impairment.

5. (canceled)

6. The method of claim 3, wherein disease/disorder is selected from disorders of the eye including wet age-related macular degeneration, dry age-related macular degeneration, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, and Sjogren's syndrome, and/or ear disorders including sensorineural hearing loss and conductive hearing loss.

7. The method of claim 3, wherein disease/disorder is selected from pulmonary disorders including chronic obstructive pulmonary disease (COPD), idiopathic pulmonary fibrosis, pulmonary hypertension, and/or cancers including melanoma, pancreatic cancer, prostate cancer, colon cancer, and leukemia, and/or short bowel syndrome, ischemia, inflammation, cardiovascular disease, congestive heart failure, dermatological disease, inflammation, or GM2 gangliosidosis.

8. A method of treating a disorder comprising aberrant signaling of PKC, the method comprising administering to a subject in need thereof a therapeutically effective dose of a compound of claim 1, wherein the disorder is selected from diabetes mellitus, diabetic nephropathy, diabetic neuropathy, diabetic retinopathy, ischemia, inflammation, pulmonary hypertension, congestive heart failure, cardiovascular disease, dermatological disease, cancer and GM2 gangliosidosis, and wherein the compound is administered in an amount of about 20 to about 200 mg once daily of the compound, or about 10 to about 100 mg twice daily.

9. (canceled)

10. A method of treating a disorder comprising aberrant signaling of RSK1, the method comprising administering to a subject in need thereof a therapeutically effective dose of a compound of claim 1, wherein the disorder is selected from breast cancer, ovarian cancer, prostate cancer, lung cancer, hepatocellular carcinoma, colorectal cancer, melanoma, osteosarcoma, leukemia, or bladder cancer, and wherein the compound is administered in an amount of about 20 to about 200 mg once daily of the compound, or about 10 to about 100 mg twice daily.

11. (canceled)

12. A method of treating a disorder comprising aberrant signaling of CDK4/6, the method comprising administering to a subject in need thereof a therapeutically effective dose of a compound of claim 1, wherein the disorder is selected from breast cancer, liposarcoma, lung cancer, glioblastoma, melanoma, pancreatic cancer, prostate cancer, colon cancer, ovarian cancer, colorectal cancer, bladder cancer, hepatocellular carcinoma, osteosarcoma, germ cell tumors, and advanced solid tumors, lymphoma, leukemia and other hematologic malignancies, and wherein the compound is administered in an amount of about 20 to about 200 mg once daily of the compound, or about 10 to about 100 mg twice daily.

13. (canceled)

14. (canceled)

15. The method of claim 3, wherein the compound is administered in combination with lithium.

16. The method of claim 3, wherein the subject is non-responsive to lithium.

17. The method of claim 3, wherein the subject is lithium responsive.

18. The method of claim 15, wherein lithium is administered at a sub-effective dose based on monotherapy, and wherein the compound is administered at a sub-effective dose based on monotherapy, wherein the sub-effective dose of lithium is about 60 mg to about 600 mg once daily, or about 30 mg to about 300 mg twice daily, and wherein a sub-effective dose of the compound is administered in about 8 to about 40 mg once daily, or about 4 to about 20 mg twice daily.

19. (canceled)

20. (canceled)

21. A method of establishing a diagnosis of bipolar disorder or other condition where GSK-3 inhibition is clinically useful, comprising administering to a subject to be evaluated a therapeutically effective dose of the compound and evaluating the subject's clinical response.

22. A method of establishing an appropriate therapeutic dose of the compound in a subject, comprising administering increasing doses of compound and assessing response using GSK-3 imaging or GSK-3 serology.

23. A method of treating a subject with Alzheimer's disease, bipolar disorder, or depression, who shows evidence of elevated GSK-3, comprising administering to the subject a therapeutically effective dose of a compound of claim 1 and evaluating and monitoring the subject using positron emission tomography (PET) or serology.

24. A method of establishing a diagnosis of bipolar disorder or other condition where GSK-3 inhibition is clinically useful, comprising administering to a subject to be evaluated a therapeutically effective dose of a compound of claim 1 with a therapeutically effective dose of lithium, and evaluating the subject's clinical response, wherein the dose of both the compound and lithium are sub-effective based on monotherapy.

25. (canceled)

26. A method of treating a subject with Alzheimer's disease, bipolar disorder, or depression, who has evidence of elevated GSK-3 beta activity, comprising administering to the subject a therapeutically effective dose of a compound of claim 1 or a pharmaceutically acceptable salt, solvate, or polymorph thereof, and a therapeutically effective dose of lithium, and monitoring the subject using positron emission topography (PET) or serology, wherein the dose of both the compound and lithium are sub-effective based on monotherapy.

27. (canceled)

28. A method of establishing an appropriate therapeutic dose of a compound listed in claim 1 in a subject, comprising administering increasing doses of the compound and lithium to the subject and assessing response using positron emission topography (PET) or serology.

29. The method of claim 4, wherein the compound is given in combination with valproic acid, lamotrigine, quetiapine, olanzapine, risperidone, aripiprazole, lurasidone, cariprazine, asenapine, carbamazepine, or xanomeline, or with the combination of xanomeline with trospium.
